(19) United States
(12) Patent Application Publication
(10) Pub. No.: US 2026/0272802 A1
(43) Pub. Date: Sep. 17, 2026

(54) Fluid Care and/or Make-Up Composition with an Ester Oil, a Porous Spherical Silica, a Non-Porous Composite Silica, Boron Nitride, Ultramarine Blue and Titanium Dioxide

(71) Applicant: L'OREAL, PARIS (FR)

(72) Inventors: MAGALI GARREC, CHEVILLY LARUE (FR); LUDOVIC THEVENET, CHEVILLY LARUE (FR); AMANDINE COLLY, CHEVILLY LARUE (FR)

(73) Assignee: L'OREAL, Paris (FR)

(21) Appl. No.: 19/469,690

(22) PCT Filed: Mar. 6, 2024
(86) PCT No.: PCT/EP2024/055866

§ 371 Date: Sep. 26, 2025

(30) Foreign Application Priority Data
Mar. 28, 2023 (FR) 2302936

(51) Int. Cl. A61K 8/44; A61K 8/06; A61K 8/25; A61K 8/29; A61K 8/37; A61Q 1/02

(52) U.S. Cl. CPC A61K 8/442; A61K 8/062; A61K 8/25; A61K 8/29; A61K 8/37; A61Q 1/02; A61K 2800/30; A61K 2800/43; A61K 2800/62

(57) ABSTRACT

The invention relates to a fluid composition for making up and/or caring for keratin materials, in particular the skin, more particularly the face, comprising a) at least one oily, preferably continuous phase comprising at least one non-volatile hydrocarbon oil of synthetic ester type, and b) porous spherical silica particles; and c) non-porous silica composite particles; and d) boron nitride particles, and e) at least one pigment of the titanium dioxide type (CI 77891) in free form; the ratio by mass of boron nitride to titanium dioxide ranging from 0.3 to 1. It also relates to a process for making up and/or caring for keratin materials, in particular the skin, and more particularly the face, consisting in applying at least the composition described above.

The present invention relates to a fluid composition for making up and/or caring for keratin materials, in particular the skin, more particularly the face, comprising an oily phase with at least one non-volatile synthetic ester oil, at least one porous spherical silica, a non-porous composite silica, boron nitride, an ultramarine blue pigment, a titanium dioxide pigment. It also relates to a process for making up and/or caring for keratin materials with said composition.

Consumers of fluid foundations aim to transform the visual appearance of their skin, with a view to making it more homogeneous and masking its irregularities and defects. This camouflage is often achieved by virtue of the coverage provided by white titanium dioxides (CI 77891) combined with black iron oxides (CI 77499) in the search for more or less light shades or more or less dark shades: Alongside this correction, most users seek a natural effect, as if the skin were not made up. The difficulty lies in the right balance of these parameters: too much coverage can lead to a grainy appearance of the skin and further accentuate its reliefs. Too little coverage leads to disappointment about the correcting quality of the product.

The foundations on the market rely on the contribution to coverage by titanium dioxides (white pigment) and black iron oxides, in order to mask imperfections. However, this can create heterogeneities. This is because titanium dioxides have a certain propensity to enter wrinkles, fine lines, pores and other reliefs of the skin. This can quickly become visible on a macroscopic scale. In addition, with the total pigment content remaining unchanged in the formulas, the lightest shades (containing the most white pigment) encounter this problem more than the others. Thus, the coverage is accentuated, as is the heterogeneity. This is then referred to as a “cakey” effect, such as a highly unesthetic floury white coating which would be deposited on the surface of the skin and deteriorates further over the course of the day.

Lastly, to correct facial irregularities, foundations tend to be very matte, as this naturally attenuates the relief. For darker shades, containing more black pigment, black iron oxides tend to accentuate the matteness.

There therefore remains the need to create new liquid formulations for making up and/or caring for keratin materials which make it possible to correct irregularities and defects of the skin, in particular the face, by obtaining a homogeneous coverage, a natural finish, and a natural luminosity and sheen of the skin which manifests in a “healthy complexion effect” without the drawbacks mentioned above, namely without the appearance of a “cakey” effect, without the appearance of emphasizing of the reliefs of the skin such as wrinkles, fine lines, pores, and without affording an excessively matte finish. It is also sought to obtain liquid formulations that make it possible to achieve these objectives by reducing the contents of titanium dioxide without the need to use black iron oxides.

The applicant has discovered, unexpectedly, that these objectives could be achieved with a liquid composition for making up and/or caring for keratin materials, in particular the skin, more particularly the face, comprising, preferably in a physiologically acceptable medium,
a) an oily, preferably continuous phase comprising at least one non-volatile hydrocarbon oil of synthetic ester type, and
b) porous spherical silica particles; and
c) non-porous silica composite particles; and
d) boron nitride particles, and
e) at least one pigment of the titanium dioxide type (CI 77891) in free form; the ratio by mass of boron nitride to titanium dioxide ranging from 0.3 to 1.0.

This discovery forms the basis of the invention.

### SUBJECTS OF THE INVENTION

A subject of the present invention is therefore a fluid composition for making up and/or caring for keratin materials, in particular the skin, more particularly the face, comprising, preferably in a physiologically acceptable medium,
a) an oily, preferably continuous phase comprising at least one non-volatile hydrocarbon oil of synthetic ester type, and
b) porous spherical silica particles; and
c) non-porous silica composite particles; and
d) boron nitride particles, and
e) at least one pigment of the titanium dioxide type (CI 77891) in free form; the ratio by mass of boron nitride to titanium dioxide ranging from 0.3 to 1.0.

According to a particularly preferred embodiment, the composition of the invention does not contain black iron oxide (C77499).

It also relates to a process for making up and/or caring for keratin materials, in particular the skin, and more particularly the face, consisting in applying at least the composition described above.

### Definitions

In the context of the present invention, the term “keratin material” notably means the skin such as the face, the body, the hands, the cheeks, the eyelids, the contour of the eyes.

The term “physiologically acceptable” is understood to mean compatible with the skin and/or its integuments, which exhibits a pleasant color, odor and feel and which does not cause unacceptable discomfort (stinging, tautness) liable to dissuade the consumer from using this composition.

The term “fluid” is understood to mean a composition that flows under its own weight at ambient temperature and atmospheric pressure.

Advantageously, a composition has a viscosity, measured at 25° C., ranging from 0.1 to 5 Pa·s, in particular from 1 to 4 Pa·s, and particularly from 1.5 to 4 Pa·s. More preferentially, the viscosity will range from 0.5 to 5 Pa·s, in particular from 0.8 to 3 Pa·s, and particularly from 0.5 to 2 Pa·s. The viscosity is measured at 25° C., using a Rheomat RM180® viscometer from the company Lamy Rhéology, equipped with a No. 3 spindle, the measurement being performed after 10 minutes of rotation of the spindle (after which time stabilization of the viscosity and of the speed of rotation of the spindle are observed), at a shear rate of 200 min⁻¹.

The term “ambient temperature” is understood to mean 25° C.

The term “atmospheric pressure” is understood to mean 760 mmHg, i.e. 10⁵ pascals.

The term “composition comprising an oily continuous phase” is understood to mean a composition selected from one of the following two forms:
i) an anhydrous composition comprising a single liquid phase which is oily or
ii) a composition in the form of a water-in-oil emulsion.

For the purposes of the present invention, the expression “anhydrous composition” denotes any composition containing less than 5.0% by weight of water, preferably less than 2.0% by weight of water, or even less than 0.5% water, relative to its total weight, and in particular a composition which is free of water.

For the purposes of the present invention, the term “water-in-oil emulsion”, also referred to as inverse emulsion, is understood to denote any composition constituted of an oily phase in which the aqueous phase is dispersed in the form of droplets, so as to observe a mixture which is macroscopically homogeneous.

For the purposes of the present invention, the expression “composition not containing black iron oxide”, is understood to mean any composition containing less than 0.1% by weight relative to the total weight of the composition of black iron oxide in free form, namely not included in a composite pigment, and in particular a composition free of black iron oxide.

The term “pigment” is understood to mean any mineral or organic white or colored particle that is insoluble in the medium of the composition, and which is intended to color and/or opacify the composition and/or the resulting deposit.

The term “titanium dioxide pigment in free form” is understood to mean any titanium dioxide particle not bound in a composite material structure. Said titanium dioxide pigment may or may not be coated with at least one surface treatment agent.

### Oily Phase

As indicated above, the composition according to the invention comprises an oily, preferably continuous phase comprising at least one non-volatile hydrocarbon oil of synthetic ester type.

The term “ester” is understood to mean any compound comprising within its structure at least one —C(═O)—O—R group, R being the residue of a carboxylic acid which has reacted with a hydroxyl function (OH) of a monoalcohol or of a polyol.

The term “synthetic ester” is understood to mean any ester compound which is not natural or of natural origin and which is obtained via a chemical route.

A “natural” compound is understood to mean a compound obtained directly from the earth or the soil, or from plants or animals, via, where appropriate, one or more physical processes, such as for example milling, refining, distillation, purification or filtration, or else obtained from a biotechnological process, in particular obtained from microbiological or cell cultures, for example from fungi or from bacteria.

A compound “of natural origin” is understood to mean a natural compound that has undergone one or more supplementary chemical or industrial treatments, giving rise to modifications that do not affect the essential qualities of this compound and/or a compound predominantly comprising natural constituents that may or may not have undergone transformations as indicated above. As non-limiting examples of supplementary chemical or industrial treatments giving rise to modifications that do not affect the essential qualities of a natural compound, mention may be made of those permitted by the regulatory bodies such as COSMOS (Reference system for cosmetic, biological and ecological products, version 3.1 of Jun. 1, 2020), or possibly defined by the AFNOR standard 16-128.

The term “oily phase” is understood to mean an organic phase which is liquid at 25° C. and atmospheric pressure and immiscible with water, comprising at least one oil and possibly containing ingredients which are soluble or miscible in said phase.

The term “oil” denotes compounds which are liquid and immiscible in water at 25° C. and atmospheric pressure (1.013×10⁵ Pa).

The term “non-volatile oil” is understood to mean an oil the vapor pressure of which at 25° C. and atmospheric pressure is non-zero and is less than 2.66 Pa and more particularly less than 0.13 Pa. By way of example, the vapor pressure may be measured via the static method or via the effusion method by isothermal thermogravimetry, depending on the vapor pressure (OCDE standard 104).

The term “hydrocarbon oil” is understood to mean an oil mainly containing carbon and hydrogen atoms and possibly one or more functions chosen from hydroxyl, ester, ether and carboxylic functions.

According to a preferred embodiment, the non-volatile oil or oils of synthetic ester type is/are selected from esters of saturated or unsaturated, linear or branched aliphatic C₁-C₂₆ mono- or polyacid and of saturated or unsaturated, linear or branched aliphatic C₁-C₂₆ mono- or polyalcohol, the total number of carbon atoms in said esters preferably being greater than or equal to 10, and preferably less than or equal to 30.

Preferably, the oil or oils of synthetic ester type may be selected from esters of saturated or unsaturated, linear or branched aliphatic C₂-C₂₀ polyacid and of saturated or unsaturated, linear or branched aliphatic C₂-C₂₀ monoalcohol. More preferentially, the ester oil or oils of synthetic ester type may be esters of saturated and linear aliphatic C₅-C₁₅ diacid and saturated and branched aliphatic C₂-C₅ monoalcohol.

The oil or oils of synthetic ester type may be selected from monoesters, diesters, triesters, tetraesters, polyesters and mixtures thereof.

The monoesters may have the following formula:

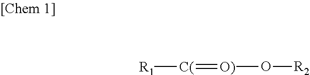

in which R₁ represents a linear or branched alkyl radical having 1 to 40 carbon atoms, preferably 7 to 19 carbon atoms, optionally comprising one or more ethylenic double bonds, and being optionally substituted, and R₂ represents a linear or branched alkyl radical having 1 to 40 carbon atoms, preferably 2 to 30 carbon atoms and more preferentially still 3 to 10 carbon atoms, optionally comprising one or more ethylenic double bonds, and being optionally substituted.

The diesters may have the following formula:

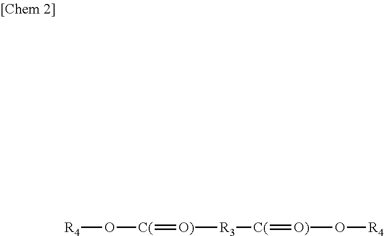

in which R₃ represents a linear or branched alkylene radical having 1 to 40 carbon atoms, preferably 7 to 19 carbon atoms, optionally comprising one or more ethylenic double bonds, and being optionally substituted, and R₄, independently at each occurrence, represents a linear or branched alkyl radical having 1 to 40 carbon atoms, preferably 3 to 30 carbon atoms and more preferentially still 3 to 10 carbon atoms, optionally comprising one or more ethylenic double bonds, and being optionally substituted.

The term “optionally substituted” is understood to mean that R₁, R₂, R₃ and/or R₄ may bear one or more substituents selected for example from groups comprising one or more heteroatoms selected from O, N and S, such as amino, amine, alkoxy and hydroxyl.

Preferably, the total number of carbon atoms of R₁+R₂ or of R₃+R₄ can be at least 9, more preferably at least 12, more preferably at least 16, and most preferably at least 20.

Among the monoesters of monoacid and of monoalcohol, mention may be made of ethyl palmitate, ethylhexyl palmitate, isopropyl palmitate, alkyl myristates such as isopropyl myristate or ethyl myristate, isocetyl stearate, 2-ethylhexyl isononanoate, isononyl isononanoate, isodecyl neopentanoate and isostearyl neopentanoate.

Use may also be made of esters of C₄-C₂₂ dicarboxylic or tricarboxylic acid and of C₁-C₂₂ alcohol and esters of monocarboxylic, dicarboxylic or tricarboxylic acid and of non-sugar dihydroxy, trihydroxy, tetrahydroxy or pentahydroxy C₄-C₂₆ alcohol.

Mention may in particular be made of: diethyl sebacate; isopropyl lauroyl sarcosinate; diisopropyl sebacate; bis(2-ethylhexyl) sebacate; diisopropyl adipate; di-n-propyl adipate; dioctyl adipate; bis(2-ethylhexyl) adipate; diisostearyl adipate; bis(2-ethylhexyl) maleate; triisopropyl citrate; triisocetyl citrate; triisostearyl citrate; glyceryl trilactate; glyceryl trioctanoate; trioctyldodecyl citrate; trioleyl citrate; neopentyl glycol diheptanoate; and diethylene glycol diisononanoate.

As non-volatile oil of synthetic ester type, use may be made of sugar esters and diesters of C₆-C₃₀ and preferably C₁₂-C₂₂ fatty acid. It is recalled that the term “sugar” is understood to mean oxygen-bearing hydrocarbon-based compounds containing several alcohol functions, with or without aldehyde or ketone functions, and which comprise at least 4 carbon atoms. These sugars may be monosaccharides, oligosaccharides or polysaccharides. Examples of suitable sugars that may be mentioned include sucrose (or saccharose), glucose, galactose, ribose, fucose, maltose, fructose, mannose, arabinose, xylose and lactose, and derivatives thereof, notably alkyl derivatives, such as methyl derivatives, for example methylglucose.

The sugar esters of fatty acids may be selected in particular from the group comprising esters or mixtures of esters of sugars described above and of linear or branched, saturated or unsaturated C₆-C₃₀ and preferably C₁₂-C₂₂ fatty acid. If they are unsaturated, these compounds may have one to three conjugated or non-conjugated carbon-carbon double bonds.

Use may be made of monooleates or dioleates of sucrose, glucose or methylglucose, stearates, behenates, oleopalmitates, linoleates, linolenates and and oleostearates. By way of example, mention may be made of the product sold under the name Glucate® DO by the company Amerchol, which is a methylglucose dioleate.

The non-volatile synthetic ester oils may for example be oleates, laurates, palmitates, myristates, behenates, cocoates, stearates, linoleates, linolenates, caprates and arachidonates, or mixtures thereof such as, in particular, mixed esters of oleopalmitate, oleostearate and palmitostearate, and pentaerythrityl tetraethylhexanoate.

As examples of preferred non-volatile oils of synthetic ester type, mention may be made, for example, of diisopropyl sebacate, diethyl sebacate, diisopropyl adipate, dioctyl adipate, 2-ethylhexyl hexanoate, ethyl laurate, cetyl octanoate, octyldodecyl octanoate, isodecyl neopentanoate, myristyl propionate, 2-ethylhexyl 2-ethylhexanoate, 2-ethylhexyl octanoate, 2-ethylhexyl caprylate/caprate, methyl palmitate, ethyl palmitate, isopropyl palmitate, dicaprylyl carbonate, isopropyl lauroyl sarcosinate, isononyl isononanoate, ethylhexyl palmitate, isohexyl laurate, hexyl laurate, isocetyl stearate, isopropyl isostearate, isopropyl myristate, isodecyl oleate, glyceryl tri(2-ethylhexanoate), pentaerythrityl tetra(2-ethylhexanoate) and 2-ethylhexyl succinate, and mixtures thereof.

According to a particularly preferred embodiment, the non-volatile oil of synthetic ester type is selected from diisopropyl sebacate such as the commercial product DUB DIS® (DUB SIS16) MB sold by the company Stéarinerie Dubois, isopropyl lauroyl sarcosinate such as the commercial product ELDEW SL-205® sold by the company Ajinomoto, isocetyl stearate such as the commercial product Isocetyl Stearate® (DUB SIS16®) MB sold by the company Stéarinerie Dubois, and mixtures thereof.

The content of non-volatile synthetic ester oil(s) present in the composition is preferably from 0.5% to 15% by weight, and more preferentially from 1% to 10% by weight, relative to the total weight of the composition.

The content of the oily phase preferably ranges from 18% to 50% by weight, and more preferentially from 25% to 40% by weight, relative to the total weight of the composition.

### Additional Oils

The composition of the present invention may comprise at least one additional oil in addition to the non-volatile hydrocarbon oil of synthetic ester type.

An oily phase suitable for the preparation of the compositions according to the present invention may comprise at least one additional oil selected from volatile or non-volatile hydrocarbon oils, volatile or non-volatile silicone oils, and mixtures thereof.

### Additional Volatile Hydrocarbon Oils

As examples of volatile hydrocarbon oils that may be used in the invention, mention may be made of:
hydrocarbon oils containing from 8 to 16 carbon atoms, and in particular C₈-C₁₆ isoalkanes of petroleum origin (also known as isoparaffins), for instance isododecane (also known as 2,2,4,4,6-pentamethylheptane), isodecane and isohexadecane, and for example the oils sold under the trade names Isopar® or Permethyl®, branched C₈-C₁₆ esters and isohexyl neopentanoate, and mixtures thereof. Other volatile hydrocarbon oils, such as petroleum distillates, in particular those sold under the name Shell Solt® by the company Shell, can also be used; volatile linear alkanes, such as those described in the patent application DE10 2008 012 457 from the company Cognis and for instance the one sold under the trade name Cetiol Ultimate® by the company BASF. Mention may also be made of the dodecane/tetradecane mixture in the 85/15 weight ratio sold by the company Biosynthis under the reference Vegelight 1214® and of the mixture of volatile linear C9-C12 alkanes having the INCI name: C9-12 Alkane, such as the product sold by the company Biosynthis under the reference Vegelight Silk®.

### Additional Non-Volatile Hydrocarbon Oils

As examples of additional non-volatile hydrocarbon oils that may be used in the invention, mention may be made of:
hydrocarbon oils of animal origin such as squalane;
linear or branched hydrocarbons, of mineral or synthetic origin, such as liquid paraffins and derivatives thereof, petroleum jelly, polydecenes, polybutenes, hydrogenated polyisobutene such as Parleam, or squalane;
phytostearyl esters, such as phytostearyl oleate, phytostearyl isostearate and lauroyl/octyldodecyl/phytostearyl glutamate (Ajinomoto, Eldew PS203®);
triglycerides constituted of fatty acid esters of glycerol, the fatty acids of which may in particular have chain lengths ranging from C₄ to C₃₆, and notably from C₁₈ to C₃₆, these oils possibly being linear or branched, and saturated or unsaturated; these oils may notably be heptanoic or octanoic triglycerides, wheatgerm oil, sunflower oil, grapeseed oil, sesame seed oil (820.6 g/mol), corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin oil, marrow oil, blackcurrant oil, evening primrose oil, millet oil, barley oil, quinoa oil, rye oil, safflower oil, candlenut oil, passionflower oil or musk rose oil; shea oil; or alternatively caprylic/capric acid triglycerides, for instance those sold by the company Stéarinerie Dubois or those sold under the names Miglyol 810®, 812® and 818® by the company Dynamit Nobel;
synthetic ethers containing from 10 to 40 carbon atoms such as dicaprylyl ether;
fatty alcohols containing from 12 to 26 carbon atoms, for instance octyldodecanol, 2-butyloctanol, 2-hexyldecanol, 2-undecylpentadecanol and oleyl alcohol;
dialkyl carbonates, the two alkyl chains possibly being identical or different, such as dicaprylyl carbonate sold under the name Cetiol CC® by Cognis; and mixtures thereof.

### Silicone Oils

For the purposes of the present invention, the term “silicone oil” is understood to mean an oil comprising at least one Si—O group, particularly organosiloxane.

### Volatile Silicone Oils

Volatile silicone oils that may be mentioned include polyalkylsiloxane linear volatile silicone oils with the INCI name Dimethicone with a viscosity at 25° C. ranging from 0.5 to 8 centistokes (from 0.5 to 8 mm²/s). The viscosity measurement method used in the invention to characterize the silicone oils according to the invention may be the “kinematic viscosity at 25° C. raw product CID-012-01” or even the “Ubbelohde viscosity at 25° C. DIN 51562-1 PV04001”. Mention may in particular be made of hexamethyldisiloxane, octamethyltrisiloxane, decamethyltetrasiloxane, tetradecamethylhexasiloxane, hexadecamethylheptasiloxane and dodecamethylpentasiloxane, and more particularly dodecamethylpentasiloxane such as the products sold under the trade names XIAMETER PMX-200 Silicone Fluid® 2 CST by the company Dow Corning, DM-FLUID®-2CS and KF-96L®-2CS by the company Shin-Etsu, Mirasil® DM 2 by Elkem Silicones, SeraSense SF 2® by KCC Corporation, BRB DM 2® by BRB International.

Volatile cyclic silicone oils with the INCI name Cyclomethicone that may be mentioned include hexamethylcyclotrisiloxane, octamethylcyclotetrasiloxane, decamethylcyclopentasiloxane and dodecamethylcyclohexasiloxane.

### Non-Volatile Silicone Oils

Among the non-volatile silicone oils, mention may be made of
non-phenyl silicone oils, for instance polydimethylsiloxanes (INCI name: Dimethicone); polydimethylsiloxanes comprising aliphatic groups, in particular alkyl groups, or alkoxy groups, which are pendent and/or at the end of the silicone chain; these groups each comprising from 6 to 24 carbon atoms, and more particularly caprylyl methicone, such as the commercial product Dow Corning FZ-3196® from the company Dow Corning;
phenyl silicone oils, for instance phenyl trimethicones, phenyl dimethicones, phenyltrimethylsiloxydiphenylsiloxanes, diphenyl dimethicones, diphenylmethyldiphenyltrisiloxanes, 2-phenylethyl trimethylsiloxysilicates and trimethylpentaphenyltrisiloxane, and mixtures thereof; and also mixtures of these various oils.

The additional oil or oils may be present in the composition in a content of at least 1% by weight, preferably at least 5% by weight, more preferentially at least 10% by weight, and even more preferentially at least 15% by weight, and in a content of less than or equal to 40% by weight, preferably less than or equal to 30% by weight, and more preferentially less than or equal to 20% by weight, relative to the total weight of the composition.

According to one particular form, the composition according to the invention comprises at least, as additional oil(s), at least one C₈-C₁₆ isoalkane of petroleum origin, in particular isododecane and/or at least one polyalkylsiloxane linear volatile silicone oil with the INCI name Dimethicone with a viscosity at 25° C. ranging from 0.5 to 8 mm²/s, and more particularly dodecamethylpentasiloxane.

### Porous Spherical Silica

The composition according to the invention comprises porous spherical silica particles.

For the purposes of the present invention, the term “porous particles” is understood to mean particles having a structure comprising pores or interstices. The structure of the particles may be of matrix type like a sponge. The porosity of the particles is characterized quantitatively by their specific surface area.

The term “spherical silicas” is understood to mean silica particles in the form or substantially in the form of a sphere, which are insoluble in the medium of the composition according to the invention, even at the melting point of the medium (about 100° C.). The spherical particles of porous silica of the present invention may have an average circularity of at least 0.8, and preferably of at least 0.82. The spherical particles of porous silica of the present invention may have an average circularity e of less than or equal to 1, preferably less than or equal to 0.99, more preferably less than or equal to 0.98, more preferably still less than or equal to 0.97, more preferably still less than or equal to 0.96, and most preferably less than or equal to 0.95.

The “average circularity” can be determined by an image analysis method. In particular, the “average circularity” may be an arithmetic mean of circularity obtained by image analysis of a scanning electron microscope (SEM) image of not less than 2000 aerogel particles observed at a magnification of 1000 by detection of secondary electrons using a scanning electron microscope. (SEM).

The “circularity” of each silica particle is a value determined by the following formula: C=4πS/L² in which C represents a circularity, S represents an area (projected area) of the particle in the image, and L represents a length of a periphery (perimeter) of the aerogel particle in the image. When the average circularity approaches 1, the form of each of the particles becomes more spherical.

The porous spherical silica particles may be present in an amount of at least 1% by weight, preferably at least 1.8% by weight, and in an amount of less than or equal to 10% by weight, more preferably less than or equal to 5% by weight, and most preferably less than or equal to 2% by weight, relative to the total weight of the composition.

The volume-average diameter of the porous spherical silica particles ranges generally from 0.1 μm (microns) to 40 μm (microns), preferably from 1 μm to 20 μm.

The sizes of the porous spherical silica particles may be measured by static light scattering using a commercial particle size analyzer such as the MasterSizer 2000® machine from Malvern. The data are processed on the basis of the Mie scattering theory. This theory, which is exact for isotropic particles, makes it possible to determine, in the case of non-spherical particles, an “effective” particle diameter. This theory is in particular described in the publication by Van de Hulst, H. C., Light Scattering by Small Particles, Chapters 9 and 10, Wiley, New York, 1957.

According to the invention, the porous spherical silica particles have a specific surface area of 30 to 1000 m²/g, preferably 100 to 900 m²/g.

The specific surface area per unit mass may be determined by the nitrogen absorption method, known as the BET (Brunauer-Emmett-Teller) method, described in the Journal of the American Chemical Society, vol. 60, page 309, February 1938 and corresponding to international standard ISO 5794/1 (annex D). The BET specific surface area corresponds to the total specific surface area of the particles under consideration.

According to a preferred embodiment, the porous spherical silica particles are selected from
porous spherical particles of amorphous silica that is not hydrophobically surface-treated;
porous spherical particles of amorphous and fumed silica;
porous spherical particles of hydrophobically surface-treated silica aerogel;
mixtures thereof.

The term “amorphous silica” is understood to mean a vitreous silica, namely a non-crystalline silica in which the atoms do not respect any order at medium and long distances, in contrast to a crystalline silica.

Porous Spherical Amorphous Silica that is not Hydrophobically Treated

As examples of porous spherical amorphous silica that is not hydrophobically surface-treated, use may be made of the following commercial products: Silica Beads SB-150®, SB-300® or SB 700®, preferentially SB 300® from the company Miyoshi Kasei; the Sunsphere® range from the company Asahi Glass AGC Si-Tech, especially Sunsphere H-51® or Sunsphere 12L®, Sunsphere H-201®, H-52 and H-53; Sunsil 130 8® from the company Sunjin; Spherica P-150® 0 from the company Ikeda Corporation; Sylosphere® from the company Fuji Silysia; the Silica Pearl® and Satinier® ranges from the company JGC Catalysts and Chemicals, more particularly Satinier M13® and Satinier M16® silicas, the MSS-500® silicas from the company Kobo, and more particularly MSS-500-20N®, and also Silica Shells® from the company Kobo.

According to a particular embodiment, the porous spherical particles of amorphous silica that are not hydrophobically treated according to the invention have an oil absorption capacity measured at the wet point ranging from 0.25 to 3.5 g/g, preferably from 0.5 to 1.5 g/g and better still from 0.7 to 1.3 g/g.

The absorption capacity measured at the wet point, denoted Wp, corresponds to the amount of oil which it is necessary to add to 100 g of particles in order to obtain a homogeneous paste. It is measured according to the “wet point” method or the method for determining the oil uptake of a powder described in the standard NF T 30-022. It corresponds to the amount of oil adsorbed onto the available surface of the powder and/or absorbed by the powder by measuring the wet point, described below: An amount m=2 g of powder is placed on a glass plate and then the oil (oleic acid) is added dropwise. After addition of 4 to 5 drops of oil to the powder, mixing is performed using a spatula, and addition of oil is continued until conglomerates of oil and powder have formed. From this point, the oil is added one drop at a time and the mixture is then triturated with the spatula. The addition of oil is stopped when a firm, smooth paste is obtained. This paste must be able to be spread over the glass plate without cracks or the formation of lumps. The mass in g of oil used is then noted.

The oil absorption capacity can also be measured in accordance with the standard JIS-K6217-4.

### Porous Amorphous and Fumed Silica

According to a particular embodiment, the porous spherical silica particles of the composition of the invention are amorphous and fumed.

The fumed silicas are hydrophobically surface-treated. It is in fact possible to chemically modify the surface of the silica, by chemical reaction generating a reduction in the number of silanol groups present at the surface of the silica. It is notably possible to substitute silanol groups with hydrophobic groups: a hydrophobic silica is then obtained.

The hydrophobic groups may be:
trimethylsiloxyl groups, which are notably obtained by treating fumed silica in the presence of hexamethyldisilazane. Silicas thus treated have the INCI name “Silica Silylate”. They are sold, for example, under the references Aerosil R812® by the company Degussa, and Cab-O-Sil TS-530® by the company Cabot,
dimethylsilyloxyl or polydimethylsiloxane groups, which are notably obtained by treating fumed silica in the presence of polydimethylsiloxane or dimethyldichlorosilane. Silicas thus treated have the INCI name “Silica Dimethyl Silylate” according to the CTFA (8th edition, 2000). They are sold, for example, under the references Aerosil R972® and Aerosil R974® by the company Degussa, and Cab-O-Sil TS-610® and Cab-O-Sil TS-720® by the company Cabot.

### Spherical Aerogel of Hydrophobic Silica

According to a particular embodiment, the porous spherical silicas are spherical aerogels of hydrophobic silica.

Aerogels are materials with high porosity. Here, silica aerogels refers to a solid silica with a porous structure generally obtained by replacing the medium included within the wet silica gels with air by drying them while maintaining a solid network structure of the silica. The porosity represents the amount of air contained in an apparent volume of a material via a percentage by volume. The spherical hydrophobic silica aerogel of the present invention may have a porosity of at least 60%, preferably at least 70%, and more preferably at least 80%.

The spherical aerogel of hydrophobic silica of the present invention is characterized in that the form of each of the particles is spherical. By virtue of this spherical shape, the hydrophobic silica aerogel makes it possible to obtain cosmetic compositions with good smoothness. The degree of sphericity of the hydrophobic silica aerogel can be determined by an average circularity.

In the spherical aerogel of hydrophobic silica of the present invention, the term “hydrophobicity” means that the silica aerogel particles are difficult to disperse in water. More precisely, this term means that an aerogel phase and an aqueous phase are completely separated after 1 g of silica aerogel particles and 100 g of ion-exchanged water are added to a bottle, the bottle is stirred or stirred for ten seconds or more, and the bottle is left to rest. Thus, in a particular embodiment of the present invention, the spherical hydrophobic silica aerogel does not have a water absorption property.

The spherical aerogel of hydrophobic silica that may be used according to the present invention is preferably of the silylated silica type (INCI name: Silica Silylate). More preferably, the spherical hydrophobic silica aerogel may be those described in JP-A-2014-088307, JP-A-2014-218433 or JP-A-2018-177620.

The hydrophobicity can be achieved by reacting a hydrophobicizing agent with a silanol group represented by the following formula existing on the surface of the silica: ≡Si—OH in which the symbol “≡” represents the remaining three valences of the Si atom, thus converting the silanol group into a group represented by the following formula: (≡Si—O—)₍₄₋ₙ₎SiRₙ in which n is an integer from 1 to 3; each R is independently a hydrocarbyl group; and two or more of R may be identical to or different from one another when n is 2 or more.

The hydrophobicizing agent may be a silylating agent. Thus, according to a preferred embodiment, in the spherical hydrophobic silica aerogel, the silica particles may be surface modified by silylation. As examples of silylation agents, mention may be made of a treating agent corresponding to one of the following formulae (1) to (3).

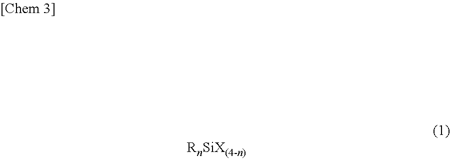

in which n represents an integer from 1 to 3; R represents a hydrocarbyl group; X represents a group that can leave a molecule via cleavage of the bond with the Si atom in a reaction with a compound having a hydroxyl group (i.e. a labile group); each R may be different when n is 2 or more; and each X may be different when n is 2 or less.

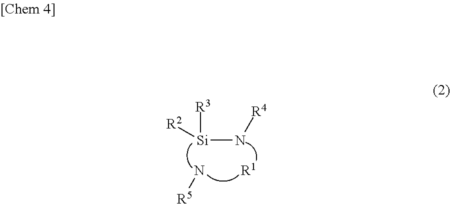

in which R¹ represents an alkylene group; R² and R³ independently represent a hydrocarbyl group; and R⁴ and R⁵ independently represent a hydrogen atom or a hydrocarbyl group.

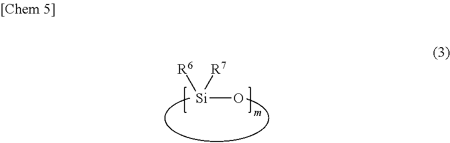

in which R⁶ and R⁷ independently represent a hydrocarbyl group; m represents an integer from 3 to 6; each R⁶ may be different when there are two or more R⁶; and each R⁷ may be different when there are two or more R⁷.

In formula (1) above, R is a hydrocarbyl group, preferably a hydrocarbyl group having a carbon number of 1 to 10, more preferably a hydrocarbyl group having a carbon number of 1 to 4, and particularly preferably a methyl group.

As examples of the labile group represented by X, mention may be made of halogen atoms such as chlorine and bromine; alkoxy groups such as a methoxy group and an ethoxy group; groups represented by —NH—SiR₃ (in which the definition of R is the same as that of R in formula (1)).

Specific examples of the hydrophobic agent represented by formula (1) above comprise: chlorotrimethylsilane, dichlorodimethylsilane, trichloromethylsilane, monomethyltrimethoxysilane, monomethyltriethoxysilane and hexamethyldisilazane.

More preferably, chlorotrimethylsilane, dichlorodimethylsilane, trichloromethylsilane and/or hexamethyldisilazane may be used from the perspective of a favorable reaction.

The number of bonds of the Si atom with the silanol group on the silica framework varies depending on the number (4-n) of the labile group X. For example, if n is 2, the following bonding will result: (≡Si—O—)₂SiR².

If n is 3, the following bonding will result: ≡Si—O—SiR₃

In this way, the silanol groups can be silylated, and thus hydrophobicization can be effected.

In formula (2) above, R¹ may be an alkylene group, preferably an alkylene group having a carbon number of 2 to 8, and particularly preferably an alkylene group having a carbon number of 2 to 3.

In formula (2) above, R² and R³ are independently a hydrocarbyl group, and the same preferred groups as those for R in formula (1) may be formed. R⁴ represents a hydrogen atom or a hydrocarbyl group, and when it is a hydrocarbyl group the same preferred groups as those for R in formula (1) may be formed. When a silica gel is treated with the compound (cyclic silazane) represented by formula (2), cleavage of the Si—N bonds will occur by reaction with silanol groups, and therefore the following bonding will result on the surface of the silica framework in the gel:

(≡Si—O—)₂SiR²R³.

In this way, the silanol group can also be silylated by the cyclic silazanes of formula (2) above, and thus hydrophobicization can be effected.

Specific examples of cyclic silazanes represented by formula (3) above include hexamethylcyclotrisilazane and octamethylcyclotetrasilazane.

In formula (3) above, R⁶ and R⁷ are independently a hydrocarbyl group, and the same preferred groups as those for R in formula (2) may be formed. m represents an integer of 3 to 6. When a silica gel is treated with the compound (cyclic siloxane) represented by formula (3), the following bonding will result on the surface of the silica framework in the gel:

(—Si—O—)₂SiR⁶R⁷.

In this way, the silanol groups can also be silylated by the cyclic siloxanes of formula (3) above, and thus hydrophobicization can be effected.

Specific examples of cyclic siloxanes represented by formula (3) above include hexamethylcyclotrisiloxane, octamethylcyclotetrasiloxane and decamethylcyclopentasiloxane.

The spherical aerogel of hydrophobic silica can be prepared by producing a silica sol, transforming the sol into gel, maturing the gel, washing the matured gel, replacing the water in the washed gel with a solvent, treating the gel with a hydrophobicizing agent, and achieving the hydrophobicization of the silica.

The spherical aerogel of hydrophobic silica may have a specific surface area determined by the BET method of at least 200 m²/g, preferably at least 400 m²/g, and more preferably at least 500 m²/g, and may have a specific surface area determined by BET method of less than or equal to 1200 m²/g, preferably less than or equal to 1000 m²/g, and more preferably less than or equal to 800 m²/g.

The spherical aerogel of hydrophobic silica may have a pore volume determined by the BJH method of at least 1 ml/g, preferably at least 2 ml/g, and more preferably at least 3 ml/g, and may have a pore volume determined by the BJH method of less than or equal to 10 ml/g, preferably less than or equal to 8 ml/g, and more preferably less than or equal to 7 ml/g.

The spherical aerogel of hydrophobic silica may have a maximum pore radius determined by the BJH method of at least 5 nm, preferably at least 10 nm, and more preferably at least 2 nm or more, and may have a maximum pore radius determined by the BJH method of less than or equal to 50 nm, preferably less than or equal to 40 nm, and more preferably less than or equal to 30 nm.

The “pore volume determined by the BJH method” refers to a pore volume which is derived from a pore having a pore radius of 1 nm to 100 nm obtained by analyzing, by the BJH method (Barrett, E. P.; Joyner, L. G.; Halenda, P. P., J. Am. Chem. Soc. 73, 373 (1951)), the adsorption isotherm on the nitrogen adsorption side obtained in the same way as explained in the “specific surface area determined by the BET method” above. The “maximum pore radius determined by the BJT method” refers to a value of a pore radius that gives a peak in a pore distribution curve (volume distribution curve) which is plotted by taking on the vertical axis the differentiation of the cumulative pore volume by the logarithm of the pore radius obtained by analyzing, by the BJH method, the adsorption isotherm on the nitrogen adsorption side obtained in the same way as above, and taking the pore radius on the horizontal axis.

The spherical aerogel of hydrophobic silica may have an average size of at least 0.5 μm, preferably at least 1 μm, and more preferably at least 2 μm, and may have an average size by image analysis method of less than or equal to 30 μm, preferably less than or equal to 20 μm, and more preferably less than or equal to 15 μm.

The “average particle size” can be measured here by an image analysis method. More precisely, the “average particle size” value is an arithmetic mean of the equivalent circle diameters that can be obtained by image analysis of a scanning electron microscope (SEM) image of, for example, not less than 2000 aerogel particles observed at a magnification of 1000 by detection of secondary electrons using a scanning electron microscope (SEM). The “equivalent circle diameter” of each aerogel particle is a diameter of a circle having a surface area equal to the surface area (projected surface area) of the aerogel particle in the image.

Preferably, the spherical aerogel of hydrophobic silica may have an oil absorption capacity, which can be measured according to the wet point method described above, of at least 2 ml/g, preferably of at least 3 ml/g, more preferably of at least 4 ml/g, and most preferably of at least 5 ml/g, and may have an oil absorption capacity, measured at the wet point, of less than or equal to 12 ml/g, preferably less than or equal to 11 ml/g, more preferably less than or equal to 10 ml/g, and most preferably less than or equal to 8 ml/g.

In a preferred embodiment of the present invention, the spherical hydrophobic silica aerogels are those described in JP-A-2014-088307, JP-A-2014-218433 or JP-A-2018-177620.

Use will more particularly be made of the spherical aerogel of hydrophobic silica sold under the trademark Airlica TL-10® by the company Tokuyama Corporation.

In a preferred embodiment, the spherical aerogel of hydrophobic silica is present in a content of at least 0.1% by weight, preferably at least 0.2% by weight, more preferably at least 0.3% by weight, and less than or equal to 1.0% by weight, preferably less than or equal to 0.8%, more preferably less than or equal to 0.7% by weight, relative to the total weight of the composition.

According to a particularly preferred form, the composition of the invention comprises a mixture of porous spherical particles of amorphous silica that is not hydrophobically treated and of porous spherical particles of silica aerogel that are hydrophobically treated as described above.

### Non-Porous Silica Composite Particles

The composition according to the present invention comprises at least non-porous silica composite particles.

In the context of the present invention, the term “silica composite particles” means silica particles within which is included at least one functional compound, preferably at least one metal oxide. Consequently, preferably, the composite silica particles may refer to “silica particles comprising at least one metal oxide”. Preferably, said metal oxide is dispersed within the silica particles.

The metal oxide may preferably be selected from titanium dioxide, zinc oxide, iron oxide and zirconium oxide, or mixtures thereof, and more particularly titanium dioxide (TiO₂) and zinc oxide (ZnO) and mixtures thereof. Particularly preferably, titanium dioxide may be used.

The non-porous silica composite particles may have an average size determined by an image analysis method of at least 0.1, preferably at least 0.5 μm, and more preferably at least 1 μm, and may have an average size by an image analysis method of less than or equal to 50 μm, preferably less than or equal to 20 μm or less, and more preferably less than or equal to 10 μm.

The “average particle size” can be determined according to the following procedure: the particle sizes are measured using an SEM image and an average value of the particle sizes is calculated.

The non-porous silica composite particles have a low oil absorption capacity.

In the non-porous silica composite particles, the ratio by weight of silica to functional compounds (preferably metal oxides and more preferably titanium dioxide) may be from 9:1 to 5:5, preferably from 4:1 to 3:2, and more preferably 7:3.

The non-porous silica composite particles may be surface treated so as to be hydrophobic. For example, the composite silica particle may be surface treated with alkylsilanes.

Most preferably, the commercial product sold under the name CHIFFONSIL-5T® (INCI name: SILICA (and) TITANIUM DIOXIDE) by the company JGC Catalysts and Chemicals may be used as non-porous silica composite particles.

The non-porous silica composite particles may be present in an amount of at least 0.5% by weight, more preferably at least 1% by weight, and most preferably at least 2% by weight, relative to the total weight of the composition. The non-porous silica composite particles are preferably present in an amount of less than or equal to 10% by weight, preferably less than or equal to 7% by weight or less, more preferably less than or equal to 5% by weight, and most preferably less than or equal to 4% by weight, relative to the total weight of the composition.

According to one particular form, the non-porous silica composite particles are present in an amount ranging from 0.7% to 1.7% by weight, and preferably ranging from 0.7% to 0.8% by weight, relative to the total weight of the composition.

### Boron Nitride

The composition according to the invention comprises boron nitride.

There are a plurality of polymorphic forms of boron nitride:
hexagonal-form boron nitrides (denoted h-BN),
rhombohedral-form boron nitrides (denoted r-BN),
amorphous-form boron nitrides (denoted a-BN),
turbostratic boron nitrides (denoted t-BN),
cubic-form boron nitrides (denoted c-BN), and
wurtzite-type hexagonal-form boron nitrides (denoted w-BN).

Hexagonal-form boron nitride h-BN has a “hexagon sheet” structure, formed by the ABAB-type stacking of BN planes which are perfectly superimposed from one plane to another by virtue of the difference in chemical nature of the elements B and N.

According to a particular form of the invention, use will be made of boron nitride particles having a platelet shape and a hexagonal form (h-BN).

Preferentially, the boron nitride particles have an oxygen content ranging from 0.05% to 3% by weight, more preferably from 0.1% to 2.5% by weight, relative to the total weight of the particle.

Preferentially, the boron nitride particles have an average particle size ranging from 0.1 to 25 μm, preferably from 0.3 to 15 μm.

The particle size is measured according to a method of distribution by laser scattering with a machine such as a Microtrac machine from Nikkiso or a 3042407 16 Mastersizer machine from Malvern, in particular by measuring the D[10], D[50] and D[90] values. D[10] represents the maximum size exhibited by 10% by volume of the particles. D[50] represents the maximum size exhibited by 50% by volume of the particles. D[90] represents the maximum size exhibited by 90% by volume of the particles.

The boron nitride particles can be modified with a surface-treatment agent making it possible to give them amphiphilic properties and to promote the dispersibility thereof in the anhydrous compositions according to the invention comprising an oily phase or in the water-in-oil emulsions additionally comprising an aqueous phase.

The boron nitride particles in accordance with the invention may be selected from the following commercial products: RonaFlair Boroneige SQ-6 ® sold by the company Merck, SP2® and SP8® sold by the company Saint Gobain Ceramics, the Softouch Boron Nitride CC6657®, CC6058®, CC6059® products sold by the company Momentive.

The ratio by mass of boron nitride to titanium dioxide ranges from 0.3 to 1.

When the ratio by mass of boron nitride to titanium dioxide is less than 0.3 or even equal to 0, a significantly elevated coverage is observed, leading to a grainy appearance of the skin and further accentuating its reliefs.

When the ratio by mass of boron nitride to titanium dioxide is greater than 1.0, insufficient coverage is observed, which does not make it possible to camouflage skin imperfections.

Preferably, a composition according to the invention comprises boron nitride in an amount ranging from 2% to 10% by weight, more preferentially from 3% to 9% by weight, and more particularly from 4% to 8% by weight, relative to the total weight of the composition.

### Ultramarine Blue Pigment

According to a preferred embodiment, the composition according to the invention additionally comprises at least one pigment of ultramarine blue type (CI 77007).

Ultramarine blue is a sulfur-containing sodium aluminum silicate with a crystal structure closely resembling that of zeolites. The color is attributed to the presence of sulfur complexes in the crystal lattice. Its INCI name is Ultramarine Blue or Ultramarines.

The pigment of ultramarine blue type may be used from the following commercial products: Bleu Outremer 09®, Ultramarine Blue 61® Azul Ultr Nubiperf H-56/Ultramarine Blue® sold by the company Venator; EC-62 Ultramarine Blue® sold by the company Nubiola; C43-1873 Suncroma Ultramarine Blue®, C43-A323 Suncroma Ultramarine Blue AR®, Suncroma Ultramarine Blue C43-1810 sold by the company Sun; Bleu Outremer Brillant W 798 Sterile®, Ultramarine Blue BC 45750®, Unipure Blue LC 680®, Cosmetic Ultramarine Blue C7104®, Ariabel Blue 300302® sold by the company Sensient; NAI-Unipure Blue LC686 sold by the company Sensient Miyoshi Kasei; Ultramarine Blue AS® from the company KS PEARL; Ultramarine Blue Cora S®, Ultramarine Blue 17® sold by the company Holliday Pigments; Cosmetic Ultramarine Blue CB-80® sold by the company Daiichi Kasei.

According to one particular form, the composition according to the invention comprises at least one ultramarine blue pigment coated with at least one lipophilic or hydrophobic compound and in particular as detailed hereinafter.

This type of pigment is particularly advantageous insofar as they exhibit a preponderant affinity for the oily gelled phase which can then convey them.

The coating may also comprise at least one additional non-lipophilic compound.

For the purposes of the invention, the “coating” of a pigment according to the invention generally denotes the total or partial surface treatment of the pigment with a surface agent, absorbed, adsorbed or grafted onto said pigment.

The surface-treated pigments may be prepared according to surface treatment techniques of chemical, electronic, mechanochemical or mechanical nature that are well known to a person skilled in the art. Commercial products may also be used.

The surface agent may be absorbed, adsorbed or grafted onto the pigments by evaporation of solvent, chemical reaction and creation of a covalent bond.

According to one variant, the surface treatment consists in coating the pigments.

The coating may represent from 0.1% to 20% by weight and in particular from 0.5% to 5% by weight, relative to the total weight of the coated pigment.

The coating may be produced, for example, by adsorption of a liquid surface agent onto the surface of the solid particles by simple mixing with stirring of the particles and of said surface agent, optionally with heating, prior to the incorporation of the particles into the other ingredients of the makeup or care composition.

The coating may be produced, for example, by chemical reaction of a surface agent with the surface of the solid pigment particles and creation of a covalent bond between the surface agent and the particles. This method is notably described in patent U.S. Pat. No. 4,578,266.

The chemical surface treatment can consist in diluting the surface agent in a volatile solvent, dispersing the pigments in this mixture and then slowly evaporating off the volatile solvent, so that the surface agent is deposited on the surface of the pigments.

When the pigment comprises a lipophilic or hydrophobic coating, it is preferably present in the fatty phase of the composition according to the invention.

According to one particular embodiment of the invention, the ultramarine blue pigments may be coated according to the invention with at least one compound selected from silicone surface agents; fluoro surface agents; fluorosilicone surface agents; metal soaps; N-acylamino acids or salts thereof; lecithin and derivatives thereof; isopropyl triisostearyl titanate; isostearyl sebacate; natural plant or animal waxes; polar synthetic waxes; fatty esters; phospholipids; and mixtures thereof.

According to a particularly preferred embodiment, the ultramarine blue pigments are coated with an N-acylamino acid and/or a salt thereof, in particular with a glutamic acid derivative and/or a salt thereof, particularly a stearoyl glutamate, such as for example aluminum stearoyl glutamate.

As examples of coated pigments according to the invention, mention may more particularly be made of the ultramarine blue pigments coated with aluminum stearoyl glutamate, for example sold under the reference NAI® by Miyoshi Kasei, in particular the product with the INCI name ULTRAMARINES (and) SILICA (and) DISODIUM STEAROYL GLUTAMATE (and) ALUMINUM HYDROXIDE sold under the trade name NAI-Unipure Blue LC686® by the company Miyoshi Kasei.

Preferably, a composition according to the invention comprises the ultramarine blue pigment in an amount ranging from 0.5% to 5% by weight, more preferentially from 0.5% to 2% by weight, relative to the total weight of the composition.

### Titanium Dioxide Pigment

The composition according to the invention comprises at least one pigment of the titanium dioxide type (CI 77891) in free form.

The size of the pigment that is useful in the context of the present invention is generally greater than 100 nm and may range up to 10 μm, preferably from 200 nm to 5 μm and more preferentially from 300 nm to 1 μm.

According to a particular form of the invention, the pigments have a size characterized by a D[50] of greater than 100 nm and possibly ranging up to 10 μm, preferably from 200 nm to 5 μm and more preferentially from 300 nm to 1 μm.

The sizes are measured by static light scattering using a commercial MasterSizer 3000 particle size analyzer from Malvern, which makes it possible to determine the particle size distribution of all of the particles over a wide range which may extend from 0.01 μm to 1000 μm. The data are processed on the basis of the standard Mie scattering theory. This theory is the most suitable for size distributions ranging from submicron to multimicron; it allows an “effective” particle diameter to be determined. This theory is in particular described in the publication by Van de Hulst, H. C., Light Scattering by Small Particles, Chapters 9 and 10, Wiley, New York, 1957. D[50] represents the maximum size exhibited by 50% by volume of the particles.

Preferably, the composition comprises at least 1% by weight of titanium dioxide pigment, more preferentially from 2% to 10% by weight, more particularly from 3% to 7% by weight, relative to the total weight of said composition.

According to one particular form, the composition according to the invention comprises at least one titanium dioxide pigment coated with at least one lipophilic or hydrophobic compound and in particular such as those detailed above.

According to a particularly preferred embodiment, the titanium dioxide pigments are coated with an N-acylamino acid and/or a salt thereof, in particular with a glutamic acid derivative and/or a salt thereof, particularly a stearoyl glutamate, such as for example aluminum stearoyl glutamate.

As examples of coated pigments according to the invention, mention may be made more particularly of titanium dioxide coated with aluminum stearoyl glutamate, for example sold under the reference NAI® by Miyoshi Kasei, in particular the product sold under the trade name NAI-White A®.

### Additional Pigments

According to one particular embodiment, the composition of the invention comprises at least one additional pigment which is different from the ultramarine blue pigments and the titanium dioxide pigments.

According to one particularly preferred form, the composition of the invention comprises at least one pigment chosen from yellow iron oxides (CI 77492), red iron oxides (CI 77491), and mixtures thereof.

Preferentially, the yellow iron oxides and/or the red iron oxides are present in amounts ranging from 0.1% to 5% by weight, more preferentially from 0.5% to 3% by weight, relative to the total weight of the composition.

According to one particular form, the yellow iron oxides and the red iron oxides used in the composition of the invention are coated with at least one lipophilic or hydrophobic compound and in particular such as those detailed above.

According to a particularly preferred embodiment, the yellow iron oxides and the red iron oxides are coated with an N-acylamino acid and/or a salt thereof, in particular with a glutamic acid derivative and/or a salt thereof, particularly a stearoyl glutamate, such as for example aluminum stearoyl glutamate.

As examples of coated yellow iron oxide and coated red iron oxide according to the invention, mention may be made more particularly of those coated with aluminum stearoyl glutamate with the INCI name: IRON OXIDES (and) DISODIUM STEAROYL GLUTAMATE (and) ALUMINUM HYDROXIDE, for example those sold under the reference NAI® by Miyoshi Kasei, in particular the product sold under the trade name NAI-C33-8001-10® (red iron oxide) and the product sold under the trade name NAI-C33-9001-10® (yellow iron oxide).

According to one particularly preferred form, the composition of the invention comprises at least one interference pigment, commonly called “pearlescent agent”.

The term “pearlescent agents” should be understood as meaning colored particles of any shape, which are or are not iridescent, in particular produced by certain mollusks in their shells or else synthesized, and which exhibit a color effect by optical interference.

The pearlescent agents may be chosen from pearlescent pigments such as titanium mica coated with an iron oxide, titanium mica coated with bismuth oxychloride, titanium mica coated with chromium oxide, titanium mica coated with an organic dye and also pearlescent pigments based on bismuth oxychloride. They may also be mica particles, at the surface of which are superposed at least two successive layers of metal oxides and/or of organic colorants.

Examples of pearlescent agents that may also be mentioned include natural mica coated with titanium oxide, with iron oxide, with tin oxide, with natural pigment and/or with bismuth oxychloride.

Among the pearlescent agents available on the market, mention may be made of the Timica, Flamenco® and Duochrome® pearlescent agents (based on mica) sold by the company BASF, the Timiron pearlescent agents sold by the company Merck, the Prestige® mica-based pearlescent agents sold by the company Eckart, and the Sunshine® synthetic mica-based pearlescent agents sold by the company Sun Chemical or Syncristal® by the company Eckart.

The pearlescent agents may more particularly have a blue, green, yellow, violet, pink, red, bronze, orangey, brown, gold and/or coppery color and/or tint.

As illustrations of pearlescent agents that may be used in the context of the present invention, mention may be made notably of the gold-colored pearlescent agents sold notably by the company BASF under the name Brilliant gold 212G® (Timica®), Gold 222C® (Cloisonné®), Sparkle Gold (Timica®), Gold 4504® (Chromalite®) and Monarch Gold 233X (Cloisonné®); the bronze pearlescent agents sold notably by the company Merck under the name Bronze Fine® (17384) (Colorona®) and Bronze® (17353) (Colorona®) and by the company BASF under the name Super Bronze® (Cloisonné®); the orange pearlescent agents sold notably by the company BASF under the name Orange 363C® (Cloisonné®) and Orange MCR 101® (Cosmica®) and by the company Merck under the name Passion Orange® (Colorona®) and Matte Orange® (17449) (Microna®); the brown pearlescent agents sold notably by the company BASF under the name Nu-antique Copper 340XB® (Cloisonné®) and Brown CL4509® (Chromalite®); the pearlescent agents with a copper tint sold notably by the company BASF under the name Copper 340A® (Timica); the pearlescent agents with a red tint sold notably by the company Merck under the name Sienna Fine® (17386) (Colorona); the pearlescent agents with a yellow tint sold notably by the company BASF under the name Yellow® (4502) (Chromalite®); the red pearlescent agents with a gold tint sold notably by the company BASF under the name Sunstone G012® (Gemtone); the pink pearlescent agents sold notably by the company BASF under the name Tan Opale G005 (Gemtone®); the black pearlescent agents with a gold tint sold notably by the company BASF under the name Nu antique bronze 240 AB® (Timica®), the blue pearlescent agents sold notably by the company Merck under the name Matte Blue® (17433) (Microna®), the white pearlescent agents with a silvery tint sold notably by the company Merck under the name Xirona Silver®, and the golden-green pink-orange pearlescent agents sold notably by the company Merck under the name Indian Summer® (Xirona®), and mixtures thereof.

Among the additional pigments that can be used according to the invention, mention may also be made of those having an optical effect different from a simple conventional coloring effect, i.e. a unified and stabilized effect such as produced by conventional colorants, for instance monochromatic pigments.

For the purposes of the invention, the term “stabilized” means lacking the effect of variability of the color with the angle of observation or in response to a temperature change.

For example, this material may be selected from particles with a metallic tint, goniochromatic coloring agents, diffractive pigments, thermochromic agents, optical brighteners, and also fibers, notably interference fibers. Needless to say, these various materials may be combined in order simultaneously to afford two effects, or even a novel effect in accordance with the invention.

The particles with a metallic tint that can be used in the invention are selected in particular from:
particles of at least one metal and/or of at least one metal derivative;
particles comprising a monomaterial or multimaterial organic or mineral substrate, at least partially coated with at least one layer with a metallic tint comprising at least one metal and/or at least one metal derivative, and mixtures of said particles.

Mention may made, among the metals which can be present in said particles, for example, of Ag, Au, Cu, Al, Ni, Sn, Mg, Cr, Mo, Ti, Zr, Pt, Va, Rb, W, Zn, Ge, Te, Se and mixtures or alloys thereof. Ag, Au, Cu, Al, Zn, Ni, Mo, Cr and mixtures or alloys thereof (for example, bronzes and brasses) are preferred metals.

The term “metal derivatives” denotes compounds derived from metals, notably oxides, fluorides, chlorides and sulfides.

Illustrations of these particles that may be mentioned include particles of aluminum, such as those sold under the names Starbrite 1200 EAC® by the company Silberline and Metalure® by the company Eckart.

Mention may also be made of the metal powders of copper or of mixtures of alloys, such as references 2844® sold by the company Radium Bronze, metal pigments, such as aluminum or bronze, such as those sold under the names Rotosafe 700® by the company Eckart, silica-coated aluminum particles sold under the name Visionaire Bright Silver® by the company Eckart and metal alloy particles, such as powders of bronze (alloy of copper and zinc) coated with silica, sold under the name Visionaire Bright Natural Gold® by the company Eckart; pearlescent agents with a white tint, in particular consisting of a synthetic mica coated with titanium dioxide and tin oxide with the INCI name SYNTHETIC FLUORPHLOGOPITE (and) TITANIUM DIOXIDE (and) TIN OXIDE, such as the product sold under the trade name Syncrystal Silk Silver® by the company Eckart.

They may also be particles comprising a glass substrate, such as those sold by the company Nippon Sheet Glass under the name Microglass Metashine®.

The goniochromatic coloring agent may be chosen, for example, from multilayer interference structures and liquid-crystal coloring agents.

Examples of symmetrical multilayer interference structures that may be used in the compositions prepared in accordance with the invention are, for example, the following structures: Al/SiO₂/Al/SiO₂/Al, pigments having this structure being sold by the company Dupont De Nemours; Cr/MgF₂/Al/MgF₂/Cr, pigments having this structure being sold under the name Chromaflair® by the company Flex; MoS₂/SiO₂/Al/SiO₂/MoS₂; Fe₂O₃/SiO₂/Al/SiO₂/Fe₂O₃, and Fe₂O₃/SiO₂/Fe₂O₃/SiO₂/Fe₂O₃, pigments having these structures being sold under the name Sicopearl® by the company BASF; MoS₂/SiO₂/mica-oxide/SiO₂/MoS₂; Fe₂O₃/SiO₂/mica-oxide/SiO₂/Fe₂O₃; TiO₂/SiO₂/TiO₂ and TiO₂/Al₂O₃/TiO₂; SnO/TiO₂/SiO₂/TiO₂/SnO; Fe₂O₃/SiO₂/Fe₂O₃; SnO/mica/TiO₂/SiO₂/TiO₂/mica/SnO, pigments having these structures being sold under the name Xirona® by the company Merck (Darmstadt). By way of example, these pigments may be the pigments of silica/titanium oxide/tin oxide structure sold under the name Xirona Magic® by the company Merck, the pigments of silica/brown iron oxide structure sold under the name Xirona Indian Summer® by the company Merck and the pigments of silica/titanium oxide/mica/tin oxide structure sold under the name Xirona Caribbean Blue® by the company Merck. Mention may also be made of the Infinite Colors® pigments from the company Shiseido. Depending on the thickness and the nature of the various layers, different effects are obtained. Thus, with the structure Fe₂O₃/SiO₂/Al/SiO₂/Fe₂O₃, the color changes from greenish gold to reddish grey for SiO₂ layers of 320 to 350 nm; from red to gold for SiO₂ layers of 380 to 400 nm; from violet to green for SiO₂ layers of 410 to 420 nm; from copper to red for SiO₂ layers of 430 to 440 nm.

As examples of pigments with a multilayer polymeric structure, mention may be made of those sold by the company 3M under the name Color Glitter®.

Examples of liquid-crystal goniochromatic particles that may be used include those sold by the company Chenix and also the product sold under the name Helicone® HC by the company Wacker.

Among the additional pigments that can be used according to the invention, mention may also be made of organic pigments.

The term “organic pigment” is understood to mean any pigment that satisfies the definition in Ullmann's Encyclopedia in the chapter on organic pigments. The organic pigment may notably be selected from nitroso, nitro, azo, xanthene, quinoline, anthraquinone, phthalocyanine, metal-complex type, isoindolinone, isoindoline, quinacridone, perinone, perylene, diketopyrrolopyrrole, thioindigo, dioxazine, triphenylmethane or quinophthalone compounds.

The additional organic pigment(s) may be selected, for example, from carmine, carbon black, aniline black, melanin, azo yellow, quinacridone, phthalocyanine blue, sorghum red, the blue pigments codified in the Color Index under the references CI 42090, 69800, 69825, 73000, 74100 and 74160, the yellow pigments codified in the Color Index under the references CI 11680, 11710, 15985, 19140, 20040, 21100, 21108, 47000 and 47005, the green pigments codified in the Color Index under the references CI 61565, 61570 and 74260, the orange pigments codified in the Color Index under the references CI 11725, 15510, 45370 and 71105, the red pigments codified in the Color Index under the references CI 12085, 12120, 12370, 12420, 12490, 14700, 15525, 15580, 15620, 15630, 15800, 15850, 15865, 15880, 17200, 26100, 45380, 45410, 58000, 73360, 73915 and 75470, and the pigments obtained by oxidative polymerization of indole or phenol derivatives as described in patent FR 2679 771.

These additional pigments may also be in the form of composite pigments as described in patent EP 1 184 426. These composite pigments may be composed in particular of particles comprising an inorganic core at least partially covered with an organic pigment and at least one binder providing the fixing of the organic pigments to the core.

The additional pigment may also be a lake. The term “lake” is understood to mean insolubilized dyes adsorbed on insoluble particles, the assembly thus obtained remaining insoluble during use.

The inorganic substrates onto which the dyes are adsorbed are, for example, alumina, silica, calcium sodium borosilicate or calcium aluminum borosilicate, and aluminum.

Among the organic dyes, mention may be made of cochineal carmine. Mention may also be made of the products known under the following names: D&C Red 21 (CI 45 380), D&C Orange 5 (CI 45 370), D&C Red 27 (CI 45 410), D&C Orange 10 (CI 45 425), D&C Red 3 (CI 45 430), D&C Red 4 (CI 15 510), D&C Red 33 (CI 17 200), D&C Yellow 5 (CI 19 140), D&C Yellow 6 (CI 15 985), D&C Green (CI 61 570), D&C Yellow 1 O (CI 77 002), D&C Green 3 (CI 42 053), D&C Blue 1 (CI 42 090).

An example of a lake that may be mentioned is the product known under the name D&C Red 7 (CI 15 850:1).

According to one particularly preferred form, for the purpose of providing reflection of light rays without losing coverage, the composition of the invention comprises at least one pearlescent agent with a white tint, in particular consisting of a synthetic mica coated with titanium dioxide and tin oxide with the INCI name SYNTHETIC FLUORPHLOGOPITE (and) TITANIUM DIOXIDE (and) TIN OXIDE, such as the product sold under the trade name Syncrystal Silk Silver by the company Eckart.

### Water-In-Oil Emulsion

According to one particular form, the composition is in the form of a water-in-oil emulsion.

The aqueous phase of a composition according to the present invention comprises at least one aqueous medium, that is to say water and optionally a water-soluble solvent.

In the present invention, the term “water-soluble solvent” denotes a compound which is liquid at ambient temperature and miscible with water (miscibility with water of greater than 50% by weight at 25° C. and atmospheric pressure).

The water-soluble solvents which can be used in the composition of the present invention may also be volatile.

Among the water-soluble solvents which can be used in the composition in accordance with the present invention, mention may be made in particular of alcohol, for example lower monoalcohols containing from 1 to 5 carbon atoms, such as ethanol and isopropanol, glycols containing from 2 to 8 carbon atoms, such as ethylene glycol, propylene glycol, 1,3-butylene glycol, and dipropylene glycol, C3 and C4 ketones, and C2-C4 aldehydes.

According to another embodiment variant, the aqueous phase of a composition according to the present invention may comprise at least one C2-C32 polyol.

For the purposes of the present invention, the term “polyol” should be understood as meaning any organic molecule comprising at least two free hydroxyl groups. Preferably, a polyol in accordance with the present invention is present in liquid form at ambient temperature.

A polyol that is suitable for use in the present invention may be a compound of linear, branched or cyclic, saturated or unsaturated alkyl type, bearing on the alkyl chain at least two —OH functions, in particular at least three —OH functions and more particularly at least four —OH functions. The polyols that are advantageously suitable for the formulation of a composition according to the present invention are those especially containing from 2 to 32 carbon atoms and preferably 3 to 16 carbon atoms. Advantageously, the polyol may be selected, for example, from ethylene glycol, pentaerythritol, trimethylolpropane, propylene glycol, 1,3-propanediol, butylene glycol, isoprene glycol, pentylene glycol, hexylene glycol, glycerol (glycerin), polyglycerols such as oligomers of glycerol, such as diglycerol, and polyethylene glycols, and mixtures thereof. According to one particular embodiment, the composition of the present invention may comprise at least glycerol.

The aqueous phase (water and optionally the water-miscible solvent) may be present in the composition in a content of at least 10% by weight, and more preferentially at least 25% by weight, and in a content of less than or equal to 50% by weight, preferably less than or equal to 45% by weight, and more preferentially less than or equal to 35% by weight, relative to the total weight of said composition.

The water-in-oil emulsions according to the invention generally comprise one or more emulsifying surfactants, which are preferably nonionic.

The emulsifier(s) is (are) present in the compositions in an amount ranging from 0.5% to 10% by weight, and more preferentially from 2% to 5% by weight, relative to the total weight of said composition.

Within the meaning of the present invention, the term “emulsifying surfactant” is understood to mean an amphiphilic surface-active compound, that is to say one exhibiting two parts of different polarities. Generally, one is lipophilic (soluble or dispersible in an oily phase). The other is hydrophilic (soluble or dispersible in water). Emulsifying surfactants are characterized by the value of their HLB (Hydrophilic Lipophilic Balance), the HLB being the ratio of the hydrophilic part to the lipophilic part in the molecule. The term “HLB” is well known to a person skilled in the art and is described, for example, in “The HLB System. A Time-Saving Guide to Emulsifier Selection” (published by ICI Americas Inc.; 1984). For emulsifying surfactants, the HLB generally ranges from 3 to 8 for the preparation of W/O emulsions. The HLB of the surfactant(s) used according to the invention can be determined by the Griffin method or the Davies method.

As examples of W/O emulsifying surfactants, mention may be made of alkyl esters or ethers of sorbitan, of glycerol, of polyol, of glycerol or of sugars; silicone surfactants, such as dimethicone copolyols, such as the product having the INCI name PEG-10 DIMETHICONE sold under the brand KF-6017® by the company Shin-Etsu; the product having the INCI name DIMETHICONE (and) PEG/PPG-18/18 DIMETHICONE sold under the brand X-22-6711 D® by the company Shin-Etsu, the product having the INCI name BIS-PEG/PPG-14/14 DIMETHICONE (and) DIMETHICONE such as the product sold under the name Abil EM 97 S® by the company Evonik Goldschmidt; the mixture of cyclomethicone and of dimethicone copolyol, sold under the name DC 5225 C® by the company Dow Corning, and alkyldimethicone copolyols such as laurylmethicone copolyol sold under the name Dow Corning 5200 Formulation Aid by the company Dow Corning; cetyl dimethicone copolyol, such as CETYL PEG/PPG-10/1 DIMETHICONE, such as the product sold under the name Abil EM 90® by the company Evonik Goldschmidt, and the mixture of cetyl dimethicone copolyol, of polyglyceryl isostearate (4 mol) and of hexyl laurate, sold under the name Abil WE 09® by the company Evonik Goldschmidt. One or more coemulsifiers, which may be selected advantageously from the group comprising polyol alkyl esters, may also be added thereto.

Mention may also be made of non-silicone emulsifying surfactants, in particular alkyl esters or ethers of sorbitan, of glycerol, of polyol or of sugars.

As polyol alkyl esters, mention may be made in particular of polyethylene glycol esters, such as PEG-30 Dipolyhydroxystearate, such as the product sold under the name Cithrol DPHS-SO-(MV)® by the company Croda.

Mention may be made, as glycerol and/or sorbitan esters, for example, of polyglyceryl isostearate (INCI name: Polyglyceryl-4 Isostearate), such as the product sold under the name Isolan GI 34® by the company Evonik Goldschmidt; sorbitan isostearate, such as the product sold under the name Arlacel 987® by the company ICI; sorbitan glyceryl isostearate, such as the product sold under the name Arlacel 986® by the company ICI, the diester of a mixture of isostearic, polyhydroxystearic and sebacic acids with Polyglycerin-4 (INCI name: Polyglyceryl-4 Diisostearate/Polyhydroxystearate/Sebacate), such as the product sold under the name Isolan GPS® by the company Evonik, and mixtures thereof.

According to a particular form of the invention, the emulsifying surfactant may be selected from emulsifying silicone elastomers.

The term “silicone elastomer” is understood to mean a flexible, deformable organopolysiloxane that has viscoelastic properties and in particular the consistency of a sponge or a flexible sphere. Its modulus of elasticity is such that this material withstands deformation and possesses a limited stretchability and contractability. This material is capable of regaining its original shape after stretching.

The emulsifying silicone elastomer may be selected from polyoxyalkylenated silicone elastomers and polyglycerolated silicone elastomers, and mixtures thereof.

### Polyoxyalkylenated Silicone Elastomers

The polyoxyalkylenated silicone elastomer is a crosslinked organopolysiloxane that may be obtained by a crosslinking addition reaction of diorganopolysiloxane containing at least one hydrogen bonded to silicon and of a polyoxyalkylene containing at least two ethylenically unsaturated groups.

Preferably, the polyoxyalkylenated crosslinked organopolysiloxane is obtained by a crosslinking addition reaction (A1) of diorganopolysiloxane containing at least two hydrogens each bonded to a silicon, and (B1) of polyoxyalkylene containing at least two ethylenically unsaturated groups, in particular in the presence (C₁) of a platinum catalyst, as described, for example, in patents U.S. Pat. Nos. 5,236,986 and 5,412,004.

In particular, the organopolysiloxane may be obtained by reaction of dimethylvinylsiloxy-terminated polyoxyalkylene (in particular polyoxyethylene and/or polyoxypropylene) and of trimethylsiloxy-terminated methylhydropolysiloxane, in the presence of a platinum catalyst.

The organic groups bonded to the silicon atoms of the compound (A1) may be alkyl groups having from 1 to 18 carbon atoms, such as methyl, ethyl, propyl, butyl, octyl, decyl, dodecyl (or lauryl), myristyl, cetyl or stearyl; substituted alkyl groups, such as 2-phenylethyl, 2-phenylpropyl or 3,3,3-trifluoropropyl; aryl groups, such as phenyl, tolyl or xylyl; substituted aryl groups, such as phenylethyl; and substituted monovalent hydrocarbon-based groups, such as an epoxy group, a carboxylate ester group or a mercapto group.

Compound (A1) may thus be selected from trimethylsiloxy-terminated methylhydropolysiloxanes, trimethylsiloxy-terminated dimethylsiloxane/methylhydrosiloxane copolymers, dimethylsiloxane/methylhydrosiloxane cyclic copolymers, and trimethylsiloxy-terminated dimethylsiloxane/methylhydrosiloxane/laurylmethylsiloxane copolymers.

Compound (C₁) is the catalyst for the crosslinking reaction, and is in particular chloroplatinic acid, chloroplatinic acid-olefin complexes, chloroplatinic acid-alkenylsiloxane complexes, chloroplatinic acid-diketone complexes, platinum black and platinum on a support.

Advantageously, the polyoxyalkylenated silicone elastomers may be formed from divinyl compounds, in particular polyoxyalkylenes containing at least two vinyl groups, reacting with Si—H bonds of a polysiloxane.

The polyoxyalkylenated silicone elastomer according to the invention is preferably mixed with at least one hydrocarbon oil and/or one silicone oil to form a gel. In these gels, the polyoxyalkylenated elastomer may be in the form of non-spherical particles.

Polyoxyalkylenated elastomers are described especially in patents U.S. Pat. Nos. 5,236,986, 5,412,004, 5,837,793 and 5,811,487.

As polyoxyalkylenated silicone elastomers, use may be made of those having the following INCI names:
Dimethicone/PEG-10/15-Crosspolymer
PEG-15/Lauryl Dimethicone Crosspolymer,
PEG-10/Lauryl Dimethicone Crosspolymer,
PEG-12 Dimethicone Crosspolymer,
PEG-10 Dimethicone Crosspolymer,
PEG-10 DimethiconeNinyl Dimethicone Crosspolymer,
PEG-12 Dimethicone/PPG-20 Crosspolymer,
and mixtures thereof.

They are especially sold under the KSG® names by the company Shin-Etsu:
KSG-210® (INCI name: Dimethicone and Dimethicone/PEG-10/15-Crosspolymer;
KSG-310® INCI name: PEG-15/Lauryl Dimethicone Crosspolymer and Mineral Oil;
KSG-320® INCI name: PEG-15/Lauryl Dimethicone Crosspolymer and Isododecane;
KSG-330® INCI name: PEG-15/Lauryl Dimethicone Crosspolymer and Triethylhexanoin;
KSG-340® INCI name: Squalane and PEG-15/Lauryl Dimethicone Crosspolymer.

They are especially sold by the company Dow Corning under the name Dow Corning 9011 Silicone Elastomer Blend®; INCI name: Cyclopentasiloxane and PEG-12 Dimethicone Crosspolymer.

Mention may also be made of the product sold under the name Dow Corning EL-7040 Hydro Elastomer Blend® by the company Dow Corning for the compound which has the INCI name: PEG-12 Dimethicone/PPG-20 Crosspolymer.

### Polyglycerolated Silicone Elastomers

The polyglycerolated silicone elastomer is an elastomeric crosslinked organopolysiloxane that may be obtained by a crosslinking addition reaction of diorganopolysiloxane containing at least one hydrogen bonded to silicon and of polyglycerolated compounds containing ethylenically unsaturated groups, especially in the presence of a platinum catalyst.

Preferably, the elastomeric crosslinked organopolysiloxane is obtained by a crosslinking addition reaction (A) of diorganopolysiloxane containing at least two hydrogens each bonded to a silicon, and (B) of glycerolated compounds containing at least two ethylenically unsaturated groups, in particular in the presence (C) of a platinum catalyst.

In particular, the organopolysiloxane may be obtained by reaction of a dimethylvinylsiloxy-terminated polyglycerolated compound and of trimethylsiloxy-terminated methylhydropolysiloxane, in the presence of a platinum catalyst.

The compound (A) is the base reactant for the formation of organopolysiloxane elastomer and the crosslinking is carried out by an addition reaction of the compound (A) with the compound (B) in the presence of the catalyst (C).

Compound (A) is in particular an organopolysiloxane containing at least two hydrogen atoms bonded to different silicon atoms in each molecule.

The compound (A) can exhibit any molecular structure, in particular a linear-chain or branched-chain structure or a cyclic structure.

Compound (A) may have a viscosity at 25° C. ranging from 1 to 50 000 centistokes, notably so as to be readily miscible with compound (B).

The organic groups bonded to the silicon atoms of the compound (A) may be alkyl groups having from 1 to 18 carbon atoms, such as methyl, ethyl, propyl, butyl, octyl, decyl, dodecyl (or lauryl), myristyl, cetyl or stearyl; substituted alkyl groups, such as 2-phenylethyl, 2-phenylpropyl or 3,3,3-trifluoropropyl; aryl groups, such as phenyl, tolyl or xylyl; substituted aryl groups, such as phenylethyl; and substituted monovalent hydrocarbon-based groups, such as an epoxy group, a carboxylate ester group or a mercapto group. Preferably, said organic group is selected from methyl, phenyl and lauryl groups.

Compound (A) may thus be selected from trimethylsiloxy-terminated methylhydropolysiloxanes, trimethylsiloxy-terminated dimethylsiloxane/methylhydrosiloxane copolymers, dimethylsiloxane/methylhydrosiloxane cyclic copolymers, and trimethylsiloxy-terminated dimethylsiloxane/methylhydrosiloxane/laurylmethylsiloxane copolymers.

Compound (B) may be a polyglycerolated compound corresponding to the formula below:

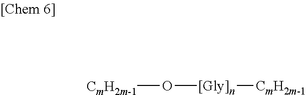

in which m is an integer ranging from 2 to 6, n is an integer ranging from 2 to 200, preferably ranging from 2 to 100, preferably ranging from 2 to 50, n preferably ranging from 2 to 20, preferably ranging from 2 to 10 and preferentially ranging from 2 to 5 and in particular equal to 3; Gly denotes: —CH₂—CH(OH)—CH₂—O— or

—CH₂—CH(CH₂OH)—O—

Advantageously, the sum of the number of ethylenic groups per molecule of the compound (B) and of the number of hydrogen atoms bonded to silicon atoms per molecule of the compound (A) is at least 4.

It is advantageous for the compound (A) to be added in an amount such that the molecular ratio of the total amount of hydrogen atoms bonded to the silicon atoms in the compound (A) to the total amount of all the ethylenically unsaturated groups in the compound (B) is within the range from 1/1 to 20/1.

The compound (C) is the catalyst of the crosslinking reaction and is in particular chloroplatinic acid, chloroplatinic acid-olefin complexes, chloroplatinic acid-alkenylsiloxane complexes, chloroplatinic acid-diketone complexes, platinum black and platinum on a support.

The catalyst (C) is preferably added from 0.1 to 1000 parts by weight and better still from 1 to 100 parts by weight, as clean platinum metal, per 1000 parts by weight of the total amount of the compounds (A) and (B).

The polyglycerolated silicone elastomer according to the invention is generally mixed with at least one hydrocarbon oil and/or one silicone oil to form a gel. In these gels, the polyglycerolated elastomer is often in the form of non-spherical particles.

Such elastomers are notably described in patent application WO 2004/024798.

Use may be made, as polyglycerolated silicone elastomers, of the following compounds having the INCI name:
Dimethicone/Polyglycerin-3 Crosspolymer,
Lauryl Dimethicone/Polyglycerin-3 Crosspolymer,
and mixtures thereof.

They are especially sold by the company Shin-Etsu under the following names:
KSG-710®; INCI name: Dimethicone/Polyglycerin-3 Crosspolymer and Dimethicone;
KSG-810®; INCI name: Mineral oil and Lauryl Dimethicone/Polyglycerin-3 Crosspolymer;
KSG-820®; INCI name: Isododecane and Lauryl Dimethicone/Polyglycerin-3 Crosspolymer;
KSG-830®; INCI name: Triethylhexanoin and Lauryl Dimethicone/Polyglycerin-3 Crosspolymer;
KSG-840®; INCI name: Squalane and Lauryl Dimethicone/Polyglycerin-3 Crosspolymer.

According to a particular form of the invention, the following will be selected as emulsifiers:
PEG-10 Dimethicone, BIS-PEG/PPG-14/14 DIMETHICONE (and) DIMETHICONE, and mixtures thereof.

### Additives

The composition according to the present invention may also comprise any other optional additives commonly used in the cosmetics field, selected for example from fillers such as magnesium sulfate, water-soluble dyes, liposoluble dyes, film-forming polymers, dispersants, antioxidants, preservatives such as phenoxyethanol or dipentaerythrityl tetrahydroxystearate/tetraisostearate, fragrances, neutralizers, pH regulators, antiseptics, active ingredients such as moisturizers, emollients, vitamins such as vitamins A, B₃, C, E and derivatives thereof, antiaging agents, organic or mineral UV screening agents or collagen-protecting agents, and mixtures thereof.

In particular, the composition according to the present invention may comprise film-forming agents such as silicone resins, for example a resin of MQ type with the INCI name TRIMETHYLSILOXYSILICATE such as the product sold under the trade name SR100® by the company Momentive Performance Materials.

The composition according to the present invention may comprise thickeners such as oily phase thickeners, for example hectorite modified with an ammonium chloride of a C10 to C22 fatty acid, in particular hectorite modified with distearyldimethylammonium chloride (INCI name: Disteardimonium Hectorite) such as the commercial product Bentone 38 VCG® from Elementis.

In addition, the composition according to the present invention may comprise additional oil absorbent particles. As examples of oil-absorbent particles, mention may be made of cellulose, silicate, perlite, magnesium carbonate, magnesium hydroxide, kaolin, talc, polyamide (in particular nylon-6), powders of acrylic polymers, in particular polymethyl methacrylate, polymethyl methacrylate/ethylene glycol dimethacrylate, polyallyl methacrylate/ethylene glycol dimethacrylate, or ethylene glycol dimethacrylate/lauryl methacrylate copolymer, silicone powders, and mixtures thereof.

These additives and the concentrations thereof should be such that they do not modify the property desired for the composition of the present invention.

### Cosmetic Compositions

The present invention also relates to a cosmetic composition comprising, in a physiologically acceptable medium, a composition as defined above.

The term “physiologically acceptable” is understood to mean compatible with the skin and/or its integuments, which exhibits a pleasant color, odor and feel and which does not cause unacceptable discomfort (stinging, tautness) liable to dissuade the consumer from using this composition.

The physiologically acceptable medium is generally suited to the nature of the support onto which the composition has to be applied, and also to the appearance under which the composition has to be packaged.

According to one particular form, the composition of the invention is in anhydrous form.

According to one particular form, the composition of the invention is in the form of a water-in-oil emulsion.

According to one particular form, the composition of the invention has a covering effect characterized by a haze ranging from 75% to 90% and a transparency (transmittance TH) of from 35% to 50%.

The covering nature of the composition is characterized by the measurement of the haze and the transparency (transmittance TH) according to the standard ASTM D 1003 (Standard Test Method for Haze and Luminous Transmittance of Transparent Plastics). Haze corresponds to the percentage of scattered light in relation to total transmittance.

Haze and transparency measurements are performed according to the following method:

25 μm films of composition were applied to 50 μm polyethylene (PE) films. The film was subsequently measured after drying for one hour at ambient temperature (25° C.). Lastly, the film is measured with a hazemeter, measuring the haze and the transparency, according to the standards ISO 13478, 14782 and ASTM D10003, D1044. The commercial reference Haze-Gard I No. 4775 from the company BYK may be used.

### APPLICATIONS

According to one embodiment, a composition of the invention can advantageously be provided in the form of a composition for caring for the skin of the body or of the face, in particular of the face.

According to another embodiment, a composition of the invention can advantageously be provided in the form of a composition for making up keratin materials, in particular the skin of the body or of the face, in particular of the face.

Thus, according to a sub-mode of this embodiment, a composition of the invention can advantageously be provided in the form of a base composition for make-up.

A composition of the invention can advantageously be provided in the form of a foundation.

Such compositions are in particular prepared according to the general knowledge of a person skilled in the art.

Throughout the description, including the claims, the term “comprising a” should be understood as being synonymous with “comprising at least one”, unless otherwise mentioned.

The invention will now be described by means of examples which are present for purely illustrative purposes and should not be interpreted as examples that limit the invention. The percentages indicated in the examples are percentages by weight relative to the total weight of the composition.

### EXAMPLES

The following example 1 according to the invention and examples 1a and 1b outside the invention were prepared.

TABLE 1

| | | | Ex1a | Ex1b |
|---|---|---|---|---|
| | | | (outside | (outside |
| | | Ex1 | the | the |
| Phase | Ingredients | (invention) | invention) | invention) |
| (A1) | PEG-10 DIMETHICONE | 3 | 3 | 3 |
| | (KF-6017 ® - Shin-Etsu) | | | |
| | BIS-PEG/PPG-14/14 DIMETHICONE | 1 | 1 | 1 |
| | (and) DIMETHICONE | | | |
| | (ABIL EM 97 S ® - Evonik | | | |
| | Goldschmidt) | | | |
| | DODECAMETHYLPENTASILOXANE, | 15 | 15 | 15 |
| | DIMETHICONE 2 CST | | | |
| | (Xiameter PMX-200 Silicone Fluid ® 2 | | | |
| | CST - Dow Corning) | | | |
| | ISOPROPYL LAUROYL | 1 | 1 | 1 |
| | SARCOSINATE | | | |
| | (Eldew SL-205 ® - Ajinomoto) | | | |
| | ISODODECANE | 14 | 14 | 14 |
| | DIISOPROPYL SEBACATE | 1 | 1 | 1 |
| | (DUB DIS ® (DUB SIS16) MB - | | | |
| | Stéarinerie Dubois) | | | |
| | DIPENTAERYTHRITYL | 0.2 | 0.2 | 0.2 |
| | TETRAHYDROXYSTEARATE/ | | | |
| | TETRAISOSTEARATE | | | |
| | (COSMOL 168 EV ® - Nisshin Oillio) | | | |
| | TOCOPHEROL | 0.1 | 0.1 | 0.1 |
| (A2) | TRIMETHYLSILOXYSILICATE | 4.7 | 4.7 | 4.7 |
| | (SR100 - Momentive Performance | | | |
| | Materials) | | | |
| (A3) | DISTEARDIMONIUM HECTORITE | 0.7 | 0.7 | 0.7 |
| | (Bentone 38 VCG ® - Elementis) | | | |
| (B) | IRON OXIDES (and) DISODIUM | 0.48 | 0.48 | 0.48 |
| | STEAROYL GLUTAMATE (and) | | | |
| | ALUMINUM HYDROXIDE/CI 77491 | | | |
| | (and) DISODIUM STEAROYL | | | |
| | GLUTAMATE (and) ALUMINUM | | | |
| | HYDROXIDE (red iron oxide) | | | |
| | (NAI-C33-8001-10 ® - Miyoshi Kasei) | | | |
| | IRON OXIDES (and) DISODIUM | 1.38 | 1.38 | 1.38 |
| | STEAROYL GLUTAMATE (and) | | | |
| | ALUMINUM HYDROXIDE/CI 77492 | | | |
| | (and) DISODIUM STEAROYL | | | |
| | GLUTAMATE (and) ALUMINUM | | | |
| | HYDROXIDE (yellow iron oxide) | | | |
| | (NAI-C33-9001-10 ® - Miyoshi Kasei) | | | |
| | IRON OXIDES (and) DISODIUM | 0.14 | 0.14 | 0.14 |
| | STEAROYL GLUTAMATE (and) | | | |
| | ALUMINUM HYDROXIDE/CI 77499 | | | |
| | (and) DISODIUM STEAROYL | | | |
| | GLUTAMATE (and) ALUMINUM | | | |
| | HYDROXIDE (black iron oxide) | | | |
| | (NAI-C33-7001-10 ® - Miyoshi Kasei) | | | |
| | TITANIUM DIOXIDE (and) | 6.8 | 12 | 0 |
| | DISODIUM STEAROYL GLUTAMATE | | | |
| | (and) ALUMINUM HYDROXIDE/CI | | | |
| | 77891 (and) DISODIUM STEAROYL | | | |
| | GLUTAMATE (and) ALUMINUM | | | |
| | HYDROXIDE | | | |
| | (NAI-White A ® - Miyoshi Kasei) | | | |
| | SYNTHETIC FLUORPHLOGOPITE | 0.8 | 0.8 | 0.8 |
| (C) | BORON NITRIDE | 5.2 | 0 | 12 |
| | (SP2 ® - Saint Gobain Ceramics) | | | |
| | (SILICA (and) TITANIUM DIOXIDE | 1 | 1 | 1 |
| | non-porous composite silica with TiO₂ | | | |
| | inclusion | | | |
| | (Chiffonsil-5T ® - JGC Catalysts and | | | |
| | Chemicals) | | | |
| | SILICA | 7.2 | 7.2 | 7.2 |
| | Amorphous porous spherical silica | | | |
| | (Sunsphere H-51 ® - Sunjin) | | | |
| | SILICA SILYLATE | 0.6 | 0.6 | 0.6 |
| | spherical porous hydrophobic silica | | | |
| | aerogel | | | |
| | (Airlica TL-10 ® - Tokuyama | | | |
| | Corporation) | | | |
| (D) | WATER | 19.5 | 19.3 | 19.3 |
| | GLYCERIN | 4 | 4 | 4 |
| | BUTYLENE GLYCOL | 4.7 | 4.7 | 4.7 |
| | MAGNESIUM SULFATE | 0.7 | 0.7 | 0.7 |
| | PHENOXYETHANOL | 0.5 | 0.5 | 0.5 |
| (E) | ALCOHOL | 6.5 | 6.5 | 6.5 |

### Protocol for the Preparation of Examples 1, 1a and 1b

The ingredients of phase (A1) described in table 1 as above described were completely mixed at ambient temperature. The ingredient of phase (A2) was added to phase (A1) and they were completely dissolved. The ingredient of phase (A3) was added and mixed with a VMI deflocculator-type homogenizer at 1500 rpm for 10 min. A mixture of the ingredients of phase (D) was added and mixed with the deflocculator for 10 min at 2500 rpm at ambient temperature. The ingredient of phase (E) was added and mixed at 1000 rpm for 15 min under cooling to 20° C. The ingredients of phases (C) and then (D) were added and were mixed at 2500 rpm for 15 min, under cooling to 20° C. The compositions obtained are foundations of the water/oil emulsion type.

### Tests of the Covering Corrective Effect

The covering corrective effect of each of the examples 1, 1a and 1 b was measured. It was characterized by haze and transparency measurements (transmittance TH). The “haze” corresponds to the percentage of light scattered with respect to the total transmittance according to standard ASTM D 1003 (Standard Test Method for Haze and Luminous Transmittance of Transparent Plastics). 25 μm films of composition were applied to 50 μm polyethylene (PE) films. The film was subsequently measured after drying for one hour at ambient temperature (25° C.). Lastly, the film was placed in the Haze-Gard I No. 4775 instrument and transparency and haze measurements were carried out. The results are collated in table 3 below.

TABLE 3

| | Transparency | Coverage |
|---|---|---|
| | (transmittance | (Contrast |
| Composition tested | T %) | Ratio CR %) |
| Example 1 (invention) | 32.17 | 78.18 |
| Example 1a | 30.25 | 74.35 |
| (outside the invention) | | |
| Free from boron nitride | | |
| Example 1b | 37.82 | 95 |
| (outside the invention) | | |
| Free from titanium | | |
| dioxide | | |

The results showed that the composition of example 1 comprising a synthetic ester oil, spherical particles of porous silica, non-porous silica composite particles, boron nitride and titanium dioxide in free form with a ratio by mass of said boron nitride to titanium dioxide ranging from 0.3 to 1.0 (0.8) gives good coverage and good transparency in contrast to the countertype 1a not containing any boron nitride and countertype 1 b not containing any titanium dioxide.

1. A fluid composition for making up and/or caring for keratin materials, in particular the skin, more particularly the face, comprising
a) at least one oily, preferably continuous phase comprising at least one non-volatile hydrocarbon oil of synthetic ester type, and
b) porous spherical silica particles; and
c) non-porous silica composite particles; and
d) boron nitride particles, and
e) at least one pigment of the titanium dioxide type (CI 77891) in free form;
the ratio by mass of boron nitride to titanium dioxide ranging from 0.3 to 1.0.

2. The composition as claimed in claim 1, not containing black iron oxide.

3. The composition as claimed in claim 1, wherein the synthetic ester oil is selected from isopropyl lauroyl sarcosinate, diisopropyl sebacate, isocetyl stearate, and mixtures thereof.

4. The composition as claimed in claim 1, wherein the content of non-volatile synthetic ester oil(s) is from 0.5% to 15% by weight relative to the total weight of the composition.

5. The composition as claimed in claim 1, comprising at least one additional oil selected from volatile or non-volatile hydrocarbon oils, volatile or non-volatile silicone oils, and mixtures thereof.

6. The composition as claimed in claim 5, wherein the additional oil or oils is/are present in a content of at least 1% by weight and in a content of less than or equal to 40% by weight relative to the total weight of the composition.

7. The composition as claimed in claim 5, comprising, as additional oil(s), at least one C8-C16 isoalkane of petroleum origin and/or at least one polyalkylsiloxane linear volatile silicone oil with the INCI name Dimethicone with a viscosity at 25° C. ranging from 0.5 to 8 mm2/s.

8. The composition as claimed in claim 1, wherein the porous spherical silica particles are selected from
porous spherical particles of amorphous silica that is not hydrophobically surface-treated;
porous spherical particles of amorphous and fumed silica;
porous spherical particles of hydrophobically surface-treated silica aerogel; and
mixtures thereof.

9. The composition as claimed in claim 1, wherein the porous spherical silica particles are present in an amount of at least 1% by weight and in an amount of less than or equal to 10% by weight relative to the total weight of the composition.

10. The composition as claimed in claim 1, wherein the non-porous spherical silica particles are present in an amount ranging from 0.7% to 1.7% by weight relative to the total weight of the composition.

11. The composition as claimed in claim 1, wherein the volume-average diameter of the porous spherical silica particles ranges generally from 0.1 to 40 μm (microns).

12. The composition as claimed in claim 1, wherein the porous spherical silica particles have a specific surface area according to the BET method of 30 to 1000 m2/g.

13. The composition as claimed in claim 9, wherein the porous spherical particles of amorphous silica that is not hydrophobically treated have an oil absorption capacity measured at the wet point ranging from 0.25 to 3.5 g/g.

14. The composition as claimed in claim 8, wherein the porous spherical hydrophobically surface-treated particles of silica aerogel have a specific surface area determined by the BET method of at least 200 m2/g, and a determined specific surface area of less than or equal to 1200 m2/g.

15. The composition as claimed in claim 8, wherein the porous spherical particles of hydrophobically surface-treated silica aerogel have a pore volume determined by the BJH method of at least 1 ml/g, and less than or equal to 10 ml/g.

16. The composition as claimed in claim 9, wherein the porous spherical particles of hydrophobically surface-treated silica aerogel have a maximum pore radius determined by the BJH method of at least 5 nm less than or equal to 50 nm.

17. The composition as claimed in claim 8, wherein the porous spherical particles of hydrophobically surface-treated silica aerogel have an oil absorption capacity, measured according to the wet point method, of at least 2 ml/g and less than or equal to 12 ml/g.

18. The composition as claimed in claim 8, wherein the porous spherical particles of hydrophobically surface-treated silica aerogel are present in a content of at least 0.1% by weight, and less than or equal to 1% by weight relative to the total weight of the composition.

19. The composition as claimed in claim 8, comprising a mixture of porous spherical particles of amorphous silica that is not hydrophobically treated and of porous spherical particles of hydrophobically treated silica aerogel.

20. The composition as claimed in claim 1, wherein the non-porous silica composite particles are silica particles in which is included at least one metal oxide.

21. The composition as claimed in claim 20, wherein the metal oxide is selected from titanium dioxide, zinc oxide, iron oxide and zirconium oxide, and mixtures thereof.

22. The composition as claimed in claim 19, wherein the non-porous silica composite particles are characterized in that the ratio by weight of silica to metal oxide is from 9:1 to 5:5.

23. The composition as claimed in claim 1, wherein the non-porous silica composite particles are present in an amount of at least 0.5% by weight and in an amount of less than or equal to 10% by weight relative to the total weight of the composition.

24. The composition as claimed in claim 1, wherein the boron nitride particles have a platelet shape and a hexagonal form (h-BN).

25. The composition as claimed in claim 1, wherein the ratio by mass of titanium dioxide to boron nitride ranges from 0.5 to 2.

26. The composition as claimed in claim 1, wherein the boron nitride is present in an amount ranging from 2% to 10% by weight relative to the total weight of the composition.

27. The composition as claimed in claim 1, additionally comprising at least one ultramarine blue pigment.

28. The composition as claimed in claim 27, wherein the ultramarine blue pigment is coated with at least one lipophilic or hydrophobic compound.

29. The composition as claimed in claim 27, wherein the ultramarine blue pigment is present in an amount ranging from 0.5% to 5% by weight relative to the total weight of the composition.

30. The composition as claimed in claim 27, comprising at least 1% by weight of titanium dioxide pigment relative to the total weight of said composition.

31. The composition as claimed in claim 1, wherein the titanium dioxide pigment is coated with at least one lipophilic or hydrophobic compound.

32. The composition as claimed in claim 30, comprising at least one additional pigment which is different from the ultramarine blue pigments and the titanium dioxide pigments.

33. The composition as claimed in claim 32, wherein the additional pigment is selected from yellow iron oxides and/or red iron.

34. The composition as claimed in claim 33, wherein the yellow iron oxides and/or the red iron oxides are present in amounts ranging from 0.1% to 5% by weight relative to the total weight of the composition.

35. The composition as claimed in claim 32, wherein the additional pigment is selected from interference pigments.

36. The composition as claimed in claim 1 in the form of a water-in-oil emulsion.

37. The composition as claimed in claim 35, wherein the aqueous phase is present in a content of at least 10% by weight and in a content of less than or equal to 50% by weight relative to the total weight of said composition.

38. The composition as claimed in claim 35, comprising one or more emulsifying surfactants.

39. The composition as claimed in claim 1 in the form of a foundation.

40. A process for making up and/or caring for keratin materials comprising applying, to said materials, at least the composition as claimed in claim 1.
